(19) United States
(12) Patent Application Publication
(10) Pub. No.: US 2009/0298795 A1
(43) Pub. Date: Dec. 3, 2009

(54) Thiol-Sensitive Positive Inotropes

(75) Inventors: Nazareno Paolocci, Baltimore, MD (US); David A. Kass, Columbia, MD (US); Carlo G. Tocchetti, Baltimore, MD (US)

Correspondence Address: MORRISON & FOERSTER LLP, 755 PAGE MILL RD, PALO ALTO, CA, 94304-1018, US

(73) Assignee: THE JOHNS HOPINS UNIVERSITY, BALTIMORE, MD (US)

(21) Appl. No.: 11/922,793

(22) PCT Filed: Jun. 23, 2006
(86) PCT No.: PCT/US2006/024545

§ 371 Date: Apr. 16, 2009

(51) Int. Cl. A61K 31/655; A61K 31/18

(52) U.S. Cl. 514/149; 514/604

(57) ABSTRACT

The present invention relates to methods for treating diastolic dysfunction or a disease, disorder or condition associated with diastolic dysfunction, methods for treating heart failure, methods for modulating SR Ca2+ release and/or uptake, methods for enhancing myocyte relaxation, preload or E2P hydrolysis, and methods for treating ventricular hypertrophy.

The present invention relates to methods for treating diastolic dysfunction or a disease, disorder or condition associated with diastolic dysfunction, methods for treating heart failure, methods for modulating SR Ca²⁺ release and/or uptake, methods for enhancing myocyte relaxation, preload or E2P hydrolysis, and methods for treating ventricular hypertrophy.

Nitroxyl (HNO), the one-electron reduced form of nitric oxide (NO), is a reactive nitrogen species with distinctive biochemical and functional properties compared to nitric oxide. Fukuto, J. M. et al., Chem. Res. Toxicol. 18, 790-801 (2005); Wink, D. A. et al., Am. J. Plysiol Heart Circ. Physiol 285, H2264-H2276 (2003). In the intact in vivo heart, the prototypic HNO donor Angeli's salt (AS) enhances cardiac function independent of β-adrenergic blockade or stimulation, and unaccompanied by changes in cGMP. Paolocci, N. et al., Proc. Natl. Acad. Sci. USA 98, 10463-10468 (2001); Paolocci, N. et al., Proc. Natl. Acad. Sci. USA 100, 5537-5542 (2003). Unlike many stimulators of contractility, HNO donors are similarly effective in normal and failing hearts. Id. Their combined ability to enhance heart function while reducing venous pressures suggests potential utility as a novel heart failure treatment.

The mechanisms underlying cardiac effects of HNO remain unknown. Recent studies suggest it can stimulate ion channels such as the NMDA receptor (Kim, W. K. et al., Neuron. 24, 461-469 (1999); Colton, C. A. et al., J. Neurochemn. 78, 1126-1134 (2001)) or skeletal muscle ryanodine receptor (Cheong, E. et al., Cell Calcium 37, 87-96 (2005). Whereas nitric oxide cardiovascular action is often coupled to cGMP, HNO action in vivo is not accompanied by changes in circulating cGMP levels. Paolocci, N. et al., Proc. Natl. Acad. Sci. USA 98, 10463-10468 (2001). However, HNO has recognized reactivity on thiols (Fukuto, J. M. et al., Chem. Res. Toxicol. 18, 790-801 (2005)) which are widely distributed as cysteine residues in proteins involved in Ca²⁺ cycling such as the SR Ca²⁺ release channel, SR Ca²⁺ pump (SERCA2a), and trans-SR membrane domain of phospholamban (PLB) (MacLennan, D. H. et al., Nat. Rev. Mol. Cell Biol. 4, 566-577 (2003).

### BRIEF DESCRIPTION OF THE DRAWINGS

FIG. 1. is a set of graphs which collectively show that HNO increases contractility and relaxation in isolated ventricular myocytes. FIG. 1A shows the effects of HNO donor AS on sarcomere shortening in isolated mouse ventricular myocytes. FIG. 1B shows dose-response effects of AS and NO donor sodium 2-(N,N-diethylamino)-diazenolate-2-oxide (DEA/NO) on sarcomere shortening in ventricular myocytes. *: p<0.001 vs. control; †: p<0.01 vs. control; **: p<0.00005 vs. control. FIG. 1C shows the effects of AS on myocyte relaxation (time to 50% relengthening). *: p<0.05 vs. control. FIG. 1D shows the kinetics of AS decomposition in Tyrode solution (pH 7.4, room temperature), and the effects of different doses of nitrite (NaNO₂) on mouse myocyte sarcomere shortening in comparison with AS/HNO. FIG. 1E shows that the nitrate produced by AS had no effect on sarcomere shortening.

FIG. 2. is a set of graphs which collectively show that AS/HNO action on myocyte function are cAMP- and cGMP-independent but modulated by the intracellular thiol content. FIG. 2A shows the kinetics of cAMP-FRET recorded in a single living neonatal rat cardiomyocyte (inset) challenged with AS (1 mM), followed by norepinephrine (NE) (10 μM) and broad-phosphodiesterase inhibitor IBMX (100 μM), and depicts FRET average over the entire cell. Summary data are to the right. *: p<10⁻⁶ vs. control. FIG. 2B shows that PKA inhibition with 100 μM Rp-CPT-cAMPs blunts isoproterenol (ISO) but not HNO inotropy. FIG. 2C shows that cGMP (ODQ) or PKG (Rp-8Br-cGMPs) inhibition blunts NO but not HNO effects. FIG. 2D shows that NO has negative impact on concomitant β-adrenergic stimulated contractility, while HNO effects are additive. FIG. 2E shows that pre-treatment with cell-permeable GSH reduces sarcomere shortening enhancement by AS/HNO. †: p<0.05 vs. control.

FIG. 3 is a set of images and graphs which collectively show the increase of Ca²⁺ transients by AS in isolated murine and rat myocytes. FIG. 3A shows linescan confocal images of Ca²⁺ transients in control and AS (0.5 mM) treated mice cardiomyocytes. Cells were loaded with Ca²⁺ indicator fluo-4 (20 μM for 20 min). Ca²⁺ transients were assessed from these scans. FIG. 3B shows mean results for Ca²⁺ transient amplitude (ΔF/F₀). FIG. 3C shows mean results for rising time (time to peak). FIG. 3D shows mean results for time from peak to 50% relaxation (T50). FIG. 3E shows basal fluorescence. n=27-28 cells from 3 hearts for each data point. *: p<0.05 vs. control, #: p<0.01 vs. control, †: p<0.001 vs. control. FIG. 3F shows representative recordings of Ca²⁺ transients in untreated (Con) and AS pretreated rat myocytes (AS). FIG. 3G and FIG. 3H show mean results for Ca²⁺ transient amplitude and τ of Ca²⁺ decline (n=30-31 cells from 4 hearts). FIG. 3I, FIG. 3J. and FIG. 3K show SR Ca²+ load measured via rapid application of 10 mM Caffeine (n=11-14 cells from 6 hearts). FIG. 3I shows twitch amplitude divided by the Caffeine amplitude expressed in % (fractional SR Ca²⁺ release). FIG. 3J shows Ca²⁺ removal fluxes according to the formula 1/τ_{twitch}=1/τ_{NCX}+1/τ_{SR}. τ_{NCX} is the τ of Ca²⁺ decline in the presence of Caffeine. Relative contribution of the SR increased from 87.6% in Con to 91.3% in AS pretreated cells, and relative contribution of NCX decreased from 12.4% to 8.7%, respectively. FIG. 3K shows that total SR load was unchanged. All data are means±SEM; *: p<0.05 vs. Con.

FIG. 4 is a set of graphs which collectively show that AS/HNO increases RyR2 function in a thiol sensitive manner and increases ATP-dependent Ca²⁺ uptake in murine sarcoplasmic reticulum (SR) vesicles. FIG. 4A shows line-scan images of Ca²⁺ sparks in intact murine myocytes in control conditions and after exposure to increased concentrations of AS/HNO. FIG. 4B shows dose-dependent effect of AS/HNO on Ca²⁺ spark frequency (left panel) (* p<0.001 vs. control), and neutral effect of the NO donor DEA/NO, at increasing concentration on Ca²⁺ spark frequency (right panel). FIG. 4C shows that pre-treatment with GSH abolishes AS-induced increase in Ca²⁺ spark frequency. FIG. 4D shows representative original tracings of single channel recordings in RyR₂ from murine myoctyes. Cardiac RyR2 channels were reconstituted into planar lipid bilayers and activated by 3 μM (cis) cytosolic Ca²⁺. From the top to the bottom, RyR2 single recordings in control conditions and after exposure to increasing concentration of AS/HNO, show dose-dependent increase in Pₒ with increasing doses of AS/HNO. In the lowest trace, the AS-induced increase in RyR2 open probability is alnost fully reversed by the addition of the thiol-reducing agent DTT to the cytosolic side. FIG. 4E shows representative stopped-flow traces of Ca²⁺ uptake obtained by subtraction of the 650 nmn (Ca-arsenazo III complex) and 693 nm (isosbestic wavelength) signals. Traces were recorded at 0.2 μM free Ca²⁺ in the presence (0.25 mM; lower trace) or absence (upper trace) of AS. Solid lines represent the best fit of a mono-exponential function plus a residual term to the stopped-flow data. FIG. 4F shows that AS significantly increased the rate constant for Ca²⁺ uptake (left panel), but did not affect the total (equilibrium) SR Ca²⁺ load (right panel).

FIG. 5 is a graph which shows the assessment method of end-diastolic pressure-volume relationship (EDPVR).

FIG. 6 is a graph which shows the effect of an NO donor nitroglycerin on EDVPR.

FIG. 7 is a set of graphs which show the effects of HNO/NO⁻ donor isopropylamine diazeniumdiolate (IPA/NO) on EDVPR. FIG. 7A shows that the HNO donated by IPA/NO produces a down-ward shift of EDPVR in chronic heart failure (CHF) preparations. FIG. 7B shows that at higher filling volumes, diastolic pressure is less in CHF hearts treated with IPA/NO vs. untreated CHF hearts.

FIG. 8 is a graph which shows mean changes in end-diastolic pressure (ΔP_{ed}) at specific LV volumes.

### DEFINITIONS

“Diastole” encompasses one or more of the following phases: isovolumic relaxation, rapid filling phase (or early diastole), slow filling phase (or diastasis), and atrial contraction. “Diastolic dysfunction” may occur when any one or more of theses phases is/are prolonged, slowed, incomplete or absent. Nonlimiting examples of diastolic dysfunction include, without limitation, the conditions described in Kass, D. A. et al., Cir. Res. 94, 1533-42 (2004); Zile M. R. et al., Prog. Cardiovasc. Dis., 47(5), 314-319 (2005); Yturralde F. R. et al., Prog. Cardiovasc. Dis., 47(5), 314-319 (2005); Owan, T. E. et a., Prog. Cardiovasc. Dis., 47(5), 320-332 (2005); Franklin, K. M. et al., Prog. Cardiovasc. Dis., 47(5), 333-339 (2005); Quiñones, M. A., Prog. Cardiovasc. Dis., 47(5), 340-355 (2005) In some embodiments, diastolic dysfunction is slowed force (or pressure) decay and cellular re-lengthening rates, increased (or decreased) early filling rates and deceleration, elevated or steeper diastolic pressure-volume (PV) relations, and/or elevated filling-rate dependent pressure.

“Disease, disorder or condition associated with diastolic dysfunction” refers to any disease, disorder or condition where diastolic dysfunction is implicated in the etiology, epidemiology, prevention and/or treatment. Nonlimiting examples include congestive heart failure, ischemic cardiomyopathy and infarction, diastolic heart failure, pulmonary congestion, pulmonary edema, cardiac fibrosis, valvular heart disease, pericardial disease, circulatory congestive states, peripheral edema, ascites, Chagas' disease, hypertension, and ventricular hypertrophy.

“Nitroxyl donor” refers to a nitroxyl (HNO) and/or nitroxyl anion (NO⁻) donating compound. Nonlimiting examples include the compounds disclosed in U.S. Pat. No. 6,936,639, US Publication No. 2004/0039063, International Publication No. WO 2005/074598, and U.S. Provisional Application No. U.S. 60/783,556, filed on Mar. 17, 2006. In some embodiments, the nitroxyl donor does not generate nitric oxide (NO).

“SR Ca²⁺ release and/or uptake” refers to calcium release from and/or uptake into the sarcoplasmic reticulum (SR)

“Preload” refers to the stretching of the myocardial cells in a chamber during diastole, prior to the onset of contraction. Preload, therefore, is related to the sarcomere length. Because sarcomere length cannot be determined in the intact heart, other indices of preload are used such as ventricular end-diastolic volume or pressure.

“Ventricular hypertrophy” includes left ventricular hypertrophy and right ventricular hypertrophy. In some embodiments, ventricular hypertrophy is left ventricular hypertrophy.

“Effective amount” refers to the amount required to produce a desired effect, for example, treating diastolic dysfunction, treating a disease, disorder or condition associated with diastolic dysfunction, treating heart failure, modulating SR Ca²⁺ release and/or uptake, enhancing myocyte relaxation, preload or E2P hydrolysis, or treating cardiac hypertrophy.

“Pharmaceutically acceptable carrier” refers to a pharmaceutically acceptable material, composition or vehicle, such as a liquid or solid filler, diluent, excipient or solvent encapsulating material, involved in carrying or transporting the subject compound from one organ, or portion of the body, to another organ or portion of the body. Each carrier is “acceptable” in the sense of being compatible with the other ingredients of the formulation and suitable for use with the patient. Examples of materials that can serve as a pharmaceutically acceptable carrier include without limitation: (1) sugars, such as lactose, glucose and sucrose; (2) starches, such as corn starch and potato starch; (3) cellulose and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

“Pharmaceutically acceptable salt” refers to an acid or base salt of the inventive compounds, which salt possesses the desired pharmacological activity and is not otherwise undesirable for administration to an animal, including a human. The salt can be formed with acids that include without limitation acetate, adipate, alginate, aspartate, benzoate, benzenesulfonate, bisulfate butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride hydrobromide, hydroiodide, 2-hydroxyethane-sulfonate, lactate, maleate, methanesulfonate, 2-naphthalenesulfonate, nicotinate, oxalate, thiocyanate, tosylate and undecanoate. Examples of a base salt include without limitation ammonium salts, alkali metal salts such as sodium and potassium salts, alkaline earth metal salts such as calcium and magnesium salts, salts with organic bases such as dicyclohexylaamine salts, N-methyl-D-glucamine, and salts with amino acids such as arginine and lysine. In some embodiments, the basic nitrogen-containing groups can be quarternized with agents including lower alkyl halides such as methyl, ethyl, propyl and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; and aralkyl halides such as phenethyl bromides.

“Isomers” refer to compounds having the same number and kind of atoms, and hence the same molecular weight, but differing with respect to the arrangement or configuration of the atoms.

“Optical isomers” includes stereoisomers, diastereoisomers and enantiomers.

“Stereoisomers” refer to isomers that differ only in the arrangement of the atoms in space.

“Diastereoisomers” refer to stereoisomers that are not mirror images of each other. Diastereoisomers occur in compounds having two or more asynmmetric carbon atoms; thus, such compounds have 2ⁿ optical isomers, where n is the number of asymmetric carbon atoms.

“Enantiomers” refer to stereoisomers that are non-superimposable mirror images of one another.

“Enantiomer-enriched” refers to a mixture in which one enantiomer predominates.

“Racemic” refers to a mixture containing equal parts of individual enantiomers.

“Non-racemic” refers to a mixture containing unequal parts of individual enantiomers.

“Animal” refers to a living organism having sensation and the power of voluntary movement, and which requires for its existence oxygen and organic food. Examples include, without limitation, members of the human, equine, porcine, bovine, murine, canine and feline species. In some embodiments, the animal is a mammal, i.e., warm-blooded vertebrate animal. In other embodiments, the animal is a human, which may also be referred to herein as “patient” or “subject”.

An animal or subject “in need of treatment” for a given disease, disorder or condition, refers to an animal or subject that is experiencing and/or is predisposed to the given disease, disorder or condition.

“Treating” refers to: (i) preventing a disease, disorder or condition from occurring in an animal that may be predisposed to the disease, disorder and/or condition but has not yet been diagnosed as having it; (ii) inhibiting a disease, disorder or condition, i.e., arresting its development; (iii) relieving a disease, disorder or condition, i.e., causing regression of the disease, disorder and/or condition; (iv) reducing severity and/or frequency of symptoms; (v) eliminating symptoms and/or underlying cause; and/or (vi) preventing the occurrence of symptoms and/or their underlying cause.

Unless the context clearly dictates otherwise, the definitions of singular terms may be extrapolated to apply to their plural counterparts as they appear in the application; likewise, the definitions of plural terms may be extrapolated to apply to their singular counterparts as they appear in the application.

### Methods of the Present Invention

Nitroxyl (HNO) is a novel redox-sensitive enhancer of heart contraction and relaxation in intact normal and failing mammalian hearts. HNO stimulates contractility and relaxation in isolated heart muscle cells by increasing the amplitude and hastening the decay of intracellular Ca²⁺ transients without altering net sarcoplasmic reticulum (SR) Ca²⁺ load or elevating rest-diastolic Ca²⁺ levels. This may result from a concomitant increase in the open probability of ryanodine-sensitive Ca²⁺ release channels, and faster Ca²⁺ re-uptake into the SR by direct stimulation of SR Ca²⁺ transport activity. These changes are independent of cAMP/PKA and cGMP/PKG, but are consistent with a HNO-thiol interaction with these proteins. The results support HNO as a novel SR-Ca²⁺ cycling enhancer with potential use in the treatment of heart failure, particularly diastolic heart failure.

Accordingly, one aspect of the present invention relates to a method for treating diastolic dysfunction or a disease, disorder or condition associated with diastolic dysfunction, comprising:
(i) identifying a subject in need of treatment for diastolic dysfunction or for a disease, disorder or condition associated with diastolic dysfunction; and
(ii) administering an effective amount of a nitroxyl donor, or a pharmaceutical composition comprising a nitroxyl donor, to the animal.

In some embodiments, the animal is a mammal. In other embodiments, the animal is a subject, i.e. human. In yet other embodiments, the subject is elderly. In yet other embodiments, the subject is female. In yet other embodiments, the subject is receiving beta-adrenergic receptor antagonist therapy. In yet other embodiments, the animal is hypertensive. In yet other embodiments, the subject is diabetic. In yet other embodiments, the subject has metabolic syndrome. In yet other embodiments, the subject has ischemic heart disease.

The nitroxyl donor may be any compound disclosed in U.S. Pat. No. 6,936,639, US Publication No. 2004/0039063, International Publication No. WO 2005/074598, and U.S. Provisional Application No. U.S. 60/783,556, filed on Mar. 17, 2006. In some embodiments, the nitroxyl donor does not generate nitric oxide (NO). In other embodiments, the nitroxyl donor is an S-nitrosothiol compound. In yet other embodiments, the nitroxyl donor is a thionitrate compound. In yet other embodiments, the nitroxyl donor is a hydroxamic acid or a pharmaceutically acceptable salt thereof. In yet other embodiments, the nitroxyl donor is a sulfohydroxamic acid or a pharmaceutically acceptable salt thereof. In yet other embodiments, the nitroxyl donor is an alkylsulfohydroxamic acid or a pharmaceutically acceptable salt thereof In yet other embodiments, the nitroxyl donor is an N-hydroxysulfonamide. In yet other embodiments, the N-hydroxysulfonamide is 2-fluoro-N-hydroxybenzenesulfonamide, 2-chloro-N-hydroxybenzenesulfonamide, 2-bromo-N-hydroxybenzenesulfonamide, 2-(trifluoromethyl)-N-hydroxybenzenesulfonamide, 5-chlorothiophene-2-sulfohydroxaric acid, 2,5-dichlorothiophene-3-sulfohydroxamic acid, 4-fluoro-N-hydroxybenzenesulfonamide, 4-trifluoro-N-hydroxybenzenesulfonamide, 4-cyano-N-hydroxybenzenesulfonamide, or 4-nitro-N-hydroxybenzenesulfonamide. In yet other embodiments, the nitroxyl donor is Piloty's acid. In yet other embodiments, the nitroxyl donor is isopropylamine diazeniumdiolate (IPA/NO). In yet other embodiments, the nitroxyl donor is Angeli's salt. Some nitroxyl donors may possess one or more asymmetric carbon center(s). As such, they may exist in the form of an optical isomer or as part of a racemic or non-racemic mixture. In some non-racemic mixtures, the R configuration may be enriched while in other non-racemic mixtures, the S configuration may be enriched.

In some embodiments, the disease, disorder or condition associated with diastolic dysfunction is diastolic heart failure. In other embodiments, the disease, disorder or condition associated with diastolic dysfunction is congestive heart failure.

Another aspect of the present invention relates to a method for treating heart failure, comprising:
(i) identifying an animal who is experiencing and/or is predisposed to impaired SR Ca²⁺ release and/or uptake, and in need of treatment for heart failure; and
(ii) administering an effective amount of a nitroxyl donor, or a pharmaceutical composition comprising a nitroxyl donor, to the animal.

Yet another aspect of the present invention relates to a method for modulating SR Ca²⁺ release and/or uptake, comprising administering an effective amount of a nitroxyl donor, or a pharmaceutical composition comprising a nitroxyl donor, to an animal in need of modulation of SR Ca²⁺ release and/or uptake.

Yet another aspect of the present invention relates to a method for enhancing myocyte relaxation, preload or E2P hydrolysis, comprising administering an effective amount of a nitroxyl donor, or a pharmaceutical composition comprising a nitroxyl donor, to an animal in need of enhancement of myocyte relaxation, preload or E2P hydrolysis.

In some embodiments, the preload is measured by end-diastolic volume (EDV). In other embodiments, the preload is measured by end-diastolic pressure (EDP).

Yet another aspect of the present invention relates to a method for treating ventricular hypertrophy, comprising administering an effective amount of a nitroxyl donor, or a pharmaceutical composition comprising a nitroxyl donor, to an animal in need of treatment of ventricular hypertrophy.

The nitroxyl donor, or pharmaceutical composition comprising a nitroxyl donor, may be administered by any means known to an ordinarily skilled artisan, for example, orally, parenterally, by inhalation spray, topically, rectally, nasally, buccally, vaginally, or via an implanted reservoir. The term “parenteral” as used herein includes subcutaneous, intravenous, intramuscular, intraperitoneal, intrathecal, intraventricular, intrastemal, intracranial, and intraosseous injection and infusion techniques.

The nitroxyl donor, or pharmaceutical composition comprising a nitroxyl donor, may be administered by a single dose, multiple discrete doses or continuous infusion. Pump means, particularly subcutaneous pump means, are useful for continuous infusion.

Dose levels on the order of about 0.001 mg/kg/d to about 10,000 mg/kg/d may be useful for the inventive methods. In some embodiments, the dose level is about 0.1 mg/kg/d to about 1,000 mg/kg/d. In other embodiments, the dose level is about 1 mg/kg/d to about 100 mg/kgld. The appropriate dose level and/or administration protocol for any given patient may vary depending upon various factors, including the activity and the possible toxicity of the specific compound employed; the age, body weight, general health, sex and diet of the patient; the time of administration; the rate of excretion; other therapeutic agent(s) combined with the compound; and the severity of the disease, disorder or condition. Typically, in vitro dosage-effect results provide useful guidance on the proper doses for patient administration. Studies in animal models are also helpful. The considerations for determining the proper dose levels and administration protocol are known to those of ordinary skill in the medical profession.

Any administration regimen well known to an ordinarily skilled artisan for regulating the timing and sequence of drug delivery can be used and repeated as necessary to effect treatment in the inventive methods. For example, the regimen may include pretreatment and/or co-administration with additional therapeutic agents. In some embodiments, the nitroxyl donor, or pharmaceutical composition comprising a nitroxyl donor, is administered alone or in combination with one or more additional therapeutic agent(s) for simultaneous, separate, or sequential use. The additional agent(s) may be any therapeutic agent(s), including without limitation one or more beta-adrenergic receptor antagonist(s) and/or compound(s) of the present invention. The nitroxyl donor, or pharmaceutical composition comprising a nitroxyl donor, may be co-administered with one or more therapeutic agent(s) either (i) together in a single formulation, or (ii) separately in individual formulations designed for optimal release rates of their respective active agent.

### Pharmaceutical Compositions of the Present Invention

Yet another aspect of the present invention relates to a pharmaceutical composition comprising:
(i) an effective amount of a compound of the present invention; and
(ii) a pharmaceutically acceptable carrier.

In some embodiments, the effective amount is the amount required to treat diastolic dysfunction. In other embodiments, the effective amount is the amount effective to treat a disease, disorder or condition associated with diastolic dysfunction. In yet other embodiments, the effective amount is the amount required to modulate SR Ca²⁺ release and/or uptake. In yet other embodiments, the effective amount is the amount required to enhance myocyte relaxation, preload or E2P hydrolysis. In yet other embodiments, the effective amount is the amount required to treat cardiac hypertrophy.

The inventive pharmaceutical compositions may comprise one or more additional pharmaceutically acceptable ingredient(s), including without limitation one or more wetting agent(s), buffering agent(s), suspending agent(s), lubricating agent(s), emulsifier(s), disintegrant(s), absorbent(s), preservative(s), surfactant(s), colorant(s), flavorant(s), sweetener(s) and additional therapeutic agent(s).

The inventive pharmaceutical composition may be formulated for administration in solid or liquid form, including those adapted for the following: (1) oral administration, for example, drenches (for example, aqueous or non-aqueous solutions or suspensions), tablets (for example, those targeted for buccal, sublingual and systemic absorption), boluses, powders, granules, pastes for application to the tongue, hard gelatin capsules, soft gelatin capsules, mouth sprays, emulsions and microemulsions; (2) parenteral administration, for example, by subcutaneous, intramuscular, intravenous or epidural injection as, for example, a sterile solution or suspension, or a sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

It will be apparent to one of ordinary skill in the art that specific embodiments of the present invention may be directed to one, some or all of the above-indicated aspects, and may encompass one, some or all of the above- and below-indicated embodiments, as well as other embodiments.

### EXAMPLES

The following examples are illustrative of the present invention and are not intended to be limitations thereon.

To determine the mechanisms of HNO cardiac activity, the present inventors assessed heart muscle cell calcium signalling and functional responses to the HNO donor, Angeli's salt, and found a novel enhancement of net SR calcium cycling independent of cAMP/PKA or cGMP but related to thiol modification.

Unless otherwise indicated, all data are presented as mean±SEM. Comparison within groups were made by Student t test, and values of p<0.05 were taken to indicate statistical significance.

### Example 1

### Effect of HNO/NO⁻ on Contractility and Relaxation in Isolated Mouse Ventricular Myocytes

### Reagensts

HNO was generated from AS (Na₂N₂O₃) that was provided by Dr. J. M. Fukuto, and NO from diethylamine (DEA)/NO that was purchased from Calbiochem/EMD Biosciences (San Diego, Calif., USA). Indo 1-AM was purchased from Molecular Probes Inc.-Invitrogen (Carlsbad, Calif., USA). ODQ was obtained from Tocris (Ellisville, Mo., USA). All other compounds were purchased from Sigma Chemical Co. (Saint Louis, Mo., USA; Milan, Italy).

### Measurenzents of Contraction and Whole Ca²⁺ Transients in Isolated Mouse Ventricular Myocytes

Wild type 2-4 month old mice were anesthetized with intraperitoneal pentobarbital sodium (100 mg/kg/ip). Hearts were perfused as previously described. Mongillo, M. et al., Circ. Res., 98, 226-234 (2006). To assess for sarcomere shortening, cells were imaged using field stimulation (Warner instruments) in an inverted fluorescence microscope (Diaphot 200; Nikon, Inc). Sarcomere length was measured by real-time Fourier transform (IonOptix MyoCam, CCCD100M) and cell twitch amplitude is expressed as a percentage of resting cell length. Twitch kinetics was quantified by measuring the time to peak shortening and the time from peak shortening to 50% relaxation. For whole calcium transient measurements, myocytes were loaded with the Ca²⁺ indicator fluo-4/AM (Molecular Probes, 20 μM for 30 min) and Ca²⁺ transients were measured under field-stimulation at 0.5 Hz in perfusion solution by confocal laser scanning microscope (LSM510, Carl Zeiss). Digital image analysis used customer-designed programs coded in Interactive Data Language (IDL).

### Results

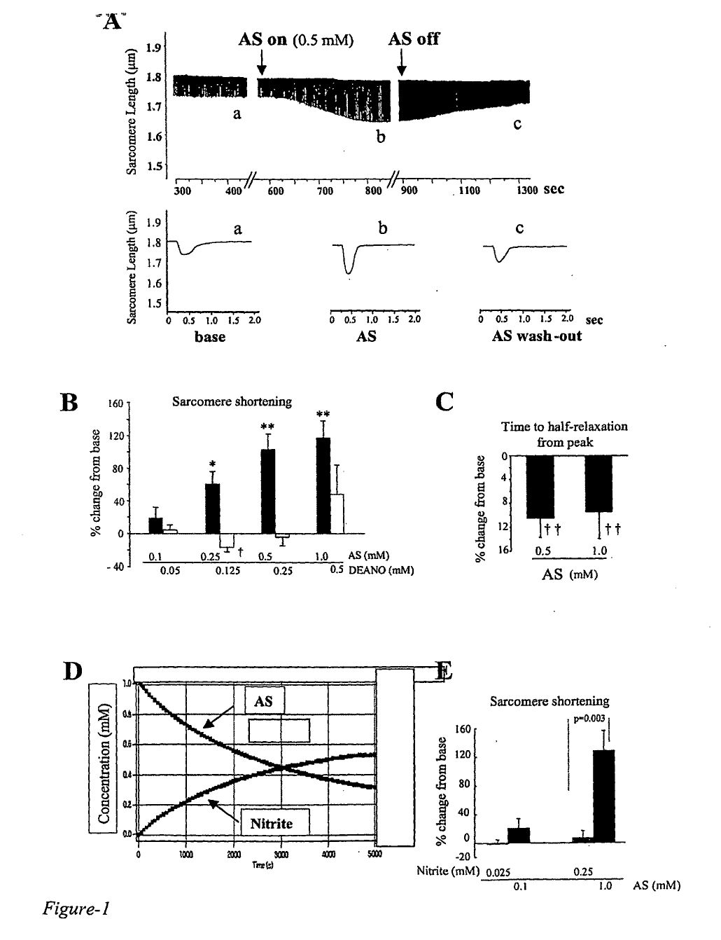

AS (10⁻⁶ to 10⁻³ M) applied to freshly isolated adult murine myocytes (C57/BL6) induced a dose-dependent increase in sarcomere shortening (FIG. 1A, FIG. 1B). Myocyte contractility rose at ≧100 μM AS, peaking at ˜100% with 0.5 and 1 mM (both p<0.00005). Myocyte relaxation also hastened by 10-20% (FIG. 1C, p<0.05). The response plateaued after ˜10-15 min, and was fully reversible after a similar time period following discontinuation at ≦500 μM (FIG. 1A). In contrast to HNO, the NO donor DEA/NO [sodium 2-(N,N-diethylamino)-diazenolate-2-oxide] induced slight depression at low doses, and minimal changes at higher doses (FIG. 1B).

At physiological pH, AS decomposes to produce HNO and nitrite. Whether nitrite could play a role in the observed responses was therefore tested. AS decomposition in the identical medium and temperature used for the myocyte studies (FIG. 1D) revealed only 25% nitrite generation after ˜1000 sec (16 min). Identical results were obtained for 0.1-1 mM AS. Thus, at time of functional analysis, 25-250 μM NO₂⁻ is expected; however, such levels (and higher or lower doses) had no effect on sarcomere shortening (FIG. 1E).

### Example 2

### Effect of cAMP and cGMP on HNO/NO⁻ Action in Isolated Rat Ventricular Myocytes

### Measuremzents of Whole Ca²⁺ Transients and SR Ca²⁺ Load in Isolated Rat Ventricular Myocytes

Isolation of ventricular myocytes from rats was carried out as previously described. Bassani, R. A. et al., J. Mol. Cell. Cardiol., 26, 1335-1347 (1994). The enzyme used for tissue dissociation was Liberase Blendzyme 3 or 4 (13-20 Wuensch Units/Heart) sometimes supplemented with 5-10 Units of Dispase II (both Roche Diagnostics, Indianapolis, Ind.). When the heart became flaccid, left ventricular tissue was cut into small pieces for further incubation (5 to 10 min at 37° C.) in enzyme solution. The tissue was dispersed, filtered, and suspensions rinsed several times before used for experiments. Isolated rat ventricular myocytes were then plated onto superfusion chambers, with the glass bottoms treated with natural mouse laminin (Invitrogen, Carlsbad, Calif.). The standard Tyrode's solution used in all experiments contained (in mM): NaCl 140, KCl 4, MgCl₂ 1, glucose 10, HEPES 5, and CaCl₂ 1, pH 7.4. Myocytes were loaded with 6 μM Indo-1/AM for 25 min and subsequently perfused for at least 30 min to allow for deesterfication of the dye. Some cells were pretreated with 0.5 mM AS (in some Caffeine experiments with 1 mM), washed and then loaded with Indo-1/AM. Concentration of the AS stock solution was verified by absorbance at 250 nm. All experiments were done at room temperature (23-25° C.) using field stimulation. Ca²⁺-transients were recorded with Clampex 8.0 and data analyzed with Clampfit.

### FRET Imaging

Primary cultures of cardiac ventricular myocytes from 1-3 days old Sprague Dawley rats (Charles River Laboratories, Wilmington, Mass.) were prepared as described. Dostal, D. E. et al., Am. J. Physiol., 263, C851-C863 (1992). Cells were transfected with a FRET-based sensor for cAMP (Zaccolo, M. et al., Science, 295, 1711-1715 (2002)) and imaged 48 hrs after transfection. During the experiments, cells were continuously perfused with HEPES buffered Ringer's modified saline (1 mmol/LCaCl₂) at room temperature. Cells were imaged on an inverted Olympus IX50 microscope upon excitation at 430 nm. Mongillo, M. et al., Circ. Res., 98, 226-234 (2006). Image analysis was performed by using ImageJ (Rasband, W. S., ImageJ, National Institutes of Health, Bethesda, Md., USA). At each time point, FRET values were measured as the 480 nm/535 nm emission ratio intensity (R) and were normalized to the 480 nm/535 nm value at time 0 s (R₀).

### Fluorescent Probes for Two-Photon Laser Scanning Microscopy and Image Acquisition

The cationic potentiometric fluorescent dye tetramethylrhodamine methyl ester (TMRM) was used to monitor changes in ΔΨₘ as previously described. Cortassa, S. et al., Biophys. J., 87, 2060-2073 (2004). The production of the fluorescent glutathione adduct GSB from the reaction of cell permeant monochlorobimane (MCB) with reduced glutathione (GSH), catalyzed by glutathione S-transferase, was used to measure intracellular glutathione levels, as described. Cortassa, S. et al., Biophys. J., 87, 2060-2073 (2004). Experimental recordings started after exposing the cardiomyocytes to an experimental Tyrode's solution. The dish containing the cardiomyocytes was equilibrated at 37° C. with unrestricted access to atmospheric oxygen on the stage of a Nikon E600FN upright microscope. Under these conditions, cells were loaded with 100 nM TMRM and 50 μM MCB for at least 20 min. The effects of AS on the intracellular GSH pool were explored in kinetics experiments performed in a flow chamber. Cardiomyocytes were exposed briefly for 3 min to 0.5 mM AS while being subjected to continuous imaging (3.5 s per image). Images were recorded using a two photon laser scanning microscope (Bio-Rad MRC-1024MP) with excitation at 740 nm (Tsunami Ti:Sa laser, Spectra-Physics). The red emission of TMRE was collected at 605±25 nm and the blue fluorescence of GSB was collected at its maximal emission (480 nm). Images were analyzed offline using ImageJ software (Wayne Rasband, National Institutes of Health, http://rsb.info.nih.gov/ij/). The statistical significance of the differences between cells in the absence or the presence of 3 mM GSH was evaluated with a t-test (small samples, unpaired t-test with two tail p-values). The normality of the data was tested with a Kolmogorov-Smirnov test.

### Results

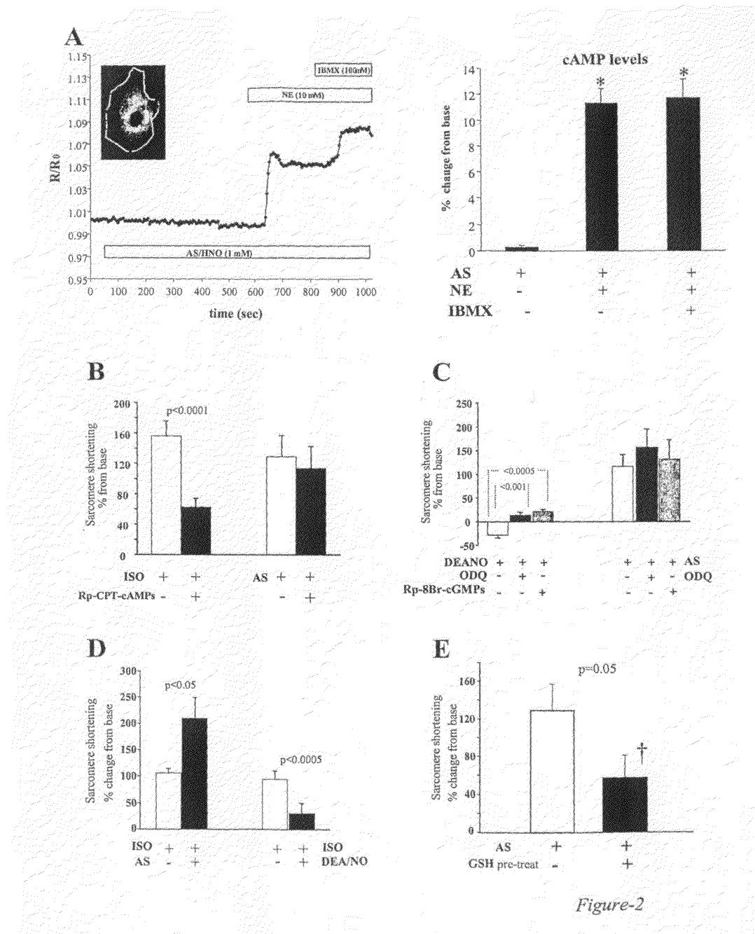

Agents that increase peak Ca²⁺ transients coupled to increased sarcomere shortening often do so via a rise in intracellular cAMP and subsequent activation of protein kinase A (PKA). Prestle, J. et al., Curr. Med. Chem., 10, 967-981 (2003). To test whether this applied to AS, real-time imaging of cAMP on transfected neonatal rat cardiomyocytes was performed with a cAMP FRET-probe. Zaccolo, M. et al., Science, 295, 1711-1715 (2002). Upon exposure to 1 mM AS, the FRET signal was unchanged (0.3%±0.1%, n=23, p=NS), whereas subsequent application of norepinephrine (10 μM) or phosphodiesterase inhibitor IBMX (100 μM) both increased it by 12% (p<10⁻⁶) (FIG. 2A). Pre-treatment of adult mouse myocytes with the PKA inhibitor Rp-CPT-cAMPs (100 μM, FIG. 2B) did not alter AS-enhanced sarcomere shortening.

AS-stimulated contractility was also independent of cGMP/PKG. Pre-incubation with the soluble guanylate cyclase inhibitor ODQ (10 μM×30 min) prevented DEA/NO-induced negative inotropy, but had no impact on AS positive inotropy. Pre-treatment with a PKG inhibitor (Rp-8Br-cGMPs, 10 μM) prevented DEA/NO negative inotropy, converting it to a modest positive response, yet had no impact on AS inotropy (FIG. 2C).

NO donors exert a negative effect on β-adrenergic stimulation in vitro and in vivo; however, the opposite has been found for HNO donors in intact hearts. Paolocci, N. et al., Proc. Natl. Acad. Sci. USA, 100, 5537-5542 (2003). The effect of HNO donors on β-adrenergic stimulation was tested in cardiomyocytes. Cells challenged with isoproterenol (ISO, 2.5 nM) had a 100±27% increase in sarcomere shortening (p=0.002, n=30). This was markedly blunted by co-infusion of 0.25 mM DEA/NO, whereas co-application of 0.5 mM AS doubled shortening above ISO alone (FIG. 2D). Thus, AS (HNO) acts in parallel with β-adrenergic stimulation pathways.

HNO targets thiol groups on selective proteins. Fukuto, J. M. et al., Chem. Res. Toxicol., 18, 790-801 (2005). To test whether such interaction could underlie whole cell contractile effects, studies were performed in which myocyte thiol equivalents were first enhanced using a cell-permeable ester-derivative of GSH (GSH ethyl ester in Tyrode's solution, 4 mM for 3 hrs). It was hypothesized that by enriching the intracellular thiol content, the probability of trapping HNO before it targeted critical thiol residues related to excitation-contraction coupling would be enhanced. Pre-treatment with GSH enhanced intracellular thiol equivalents (+6±1.5% in fluorescence a.u. vs., controls, n=40, p<0.05), as determined by fluorescence assay of glutathione S-bimane production using two-photon microscopy. Pre-treated cells were then exposed to AS (0.5 mM), and the contractility response was substantially blunted (+57±19%; p=0.02 vs. base; p=0.05 vs. AS alone) (FIG. 2E). This supports the targeting of HNO on SH groups to exert its cardiotropic action.

### Example 3

### Effect of HNO/NO⁻ on Ca²⁺ Transients in Isolated Adult Mouse and Rat Cardiac Myocytes

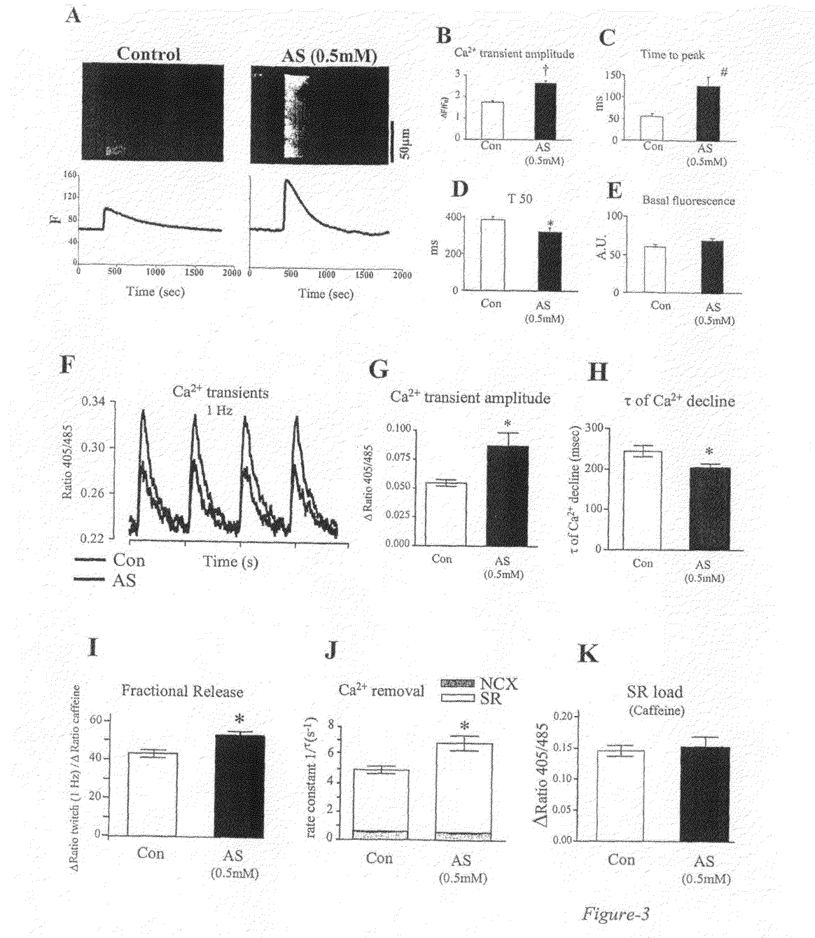

To further explore potential HNO targets, calcium cycling in adult mouse and rat cardiac myocytes was examined. Cells were first exposed to AS for 5-10 min, then washed and loaded with Indo-1 or Fluo-4 for 20 min. Pretreatment with AS was carried out because the drug reacted with the Ca²⁺ indicators (both Fluo-4 and Indo-1) and altered their fluorescent properties. In mice, the calcium transient amplitude assessed by confocal line scan imaging increased by ˜40% over baseline with 0.5 mM AS, (n=27, p<0.001) (FIG. 3A and FIG. 3B), time to peak transient was prolonged (FIG. 3C) while the decay time shortened (FIG. 3D). Basal fluorescence (F₀) was unchanged by AS pretreatment (FIG. 3E). Similar results were obtained in rat myocytes (using Indo-1) for Ca²⁺ transient amplitude (FIG. 3F and FIG. 3G) and decay time (FIG. 3H). The increase in amplitude was not accompanied by an increase in diastolic Ca²⁺ level (ratio 405/485=0.239±0.006 (Con) vs. 0.243±0.008 (AS); n.s.; see also FIG. 3A, FIG. 3E and FIG. 3F). Rapid sustained caffeine (10 mM) application abruptly releases all SR Ca²⁺ and subsequent [Ca²⁺]ᵢ decline is mediated mainly via Na/Ca exchange. The amplitude and decline of the caffeine-induced Ca²⁺ transient indicates that HNO did not alter SR Ca²⁺ content (FIG. 3K) or Na/Ca exchange fulnction (τ=2.0±0.4 vs. 2.2±0.3 s, FIG. 3J). These results indicate that HNO-enhanced [Ca²⁺]ᵢ decline was due to increased SR Ca²⁺-ATPase function, and HNO-enhanced Ca²⁺ transient amplitude was due to enhanced fractional SR Ca²⁺ release (FIG. 3I) with unaltered SR Ca²⁺ content.

### Example 4

### Effect of HNO/NO⁻ on RyR2 Function and ATP-dependent Ca²⁺ Uptake in Murine Sarcoplasmic Reticulum (SR) Vesicles

Given evidence for enhanced SR calcium re-uptake and release, with no net gain in total SR Ca²⁺ content, direct effects of HNO/NO⁻ on the ryanodine-sensitive release channel (RyR2) were examined. The effects of HNO/NO⁻ on SR membrane vesicles isolated from pooled C57/BL6 mouse hearts were also studied to test whether HNO directly enhances SR Ca²⁺ uptake.

### Visualization of Spontaneous Ca²⁺ Sparks ard Measurement of Spark Frequency

Freshly isolated mouse cardiac myocytes were loaded with the Ca²⁺ indicator fluo-4/AM (Molecular Probes, 20 μM for 30 min). Confocal images were acquired using a confocal laser-scanning microscope (LSM510, Carl Zeiss) with a Zeiss Plan-Neofluor 40× oil immersion objective (NA=1.3). Fluo-4/AM was excited by an argon laser (488 nm), and fluorescence was measured at >505 nm. Images were taken in the line-scan mode, with the scan line parallel to the long axis of the myocytes. Each image consisted of 512 line scans obtained at 1.92 ms intervals, each comprising 512 pixels at 0.10 μm separation. Digital image analysis used customer-designed programs coded in Interactive Data language (IDL) and a modified spark detection algorithm. Cheng, H. et al., Biophys. J, 76, 606-617 (1999).

### RyR2 Single Cltannel Recordings in Planar Lipid Bilayers

Recording of single RyR2 in lipid bilayers was performed as previously described. Jiang, M. T. et al., Circ. Res., 91, 1015-1022 (2002). Briefly, a phospholipid bilayer of PE:PS (1:1 dissolved in n-decane to 20 mg/ml) was formed across an aperture of ˜300 μm diameter in a delrin cup. The cis chamber (900 μl) was the voltage control side connected to the head stage of a 200A Axopatch amplifier, while the trans chamber (800 μl) was held at virtual ground. Both chambers were initially filled with 50 mM cesium methanesulfonate and 10 mM Tris/Hepes pH 7.2. After bilayer formation, cesium methanesulfonate was raised to 300 mM in the cis side and 100 to 200 μg of mouse cardiac SR vesicles was added. After detection of channel openings, Cs⁺ in the trans chamber was raised to 300 mM to collapse the chemical gradient. Single channel data were collected at steady voltages (−30 mV) for 2-5 min. Channel activity was recorded with a 16-bit VCR-based acquisition and storage system at a 10 kHz sampling rate. Signals were analyzed after filtering with an 8-pole Bessel filter at a sampling frequency of 1.5-2 kHz. Data acquisition and analysis were done with Axon Instruments software and hardware (pClamp v8.0, Digidata 200 AD/DA interface).

Isolation of (SR) Vesicles from Murine Myocardium and Measurements of ATP-dependent Ca²⁺ Uptake by Murine Cardiac SR Vesicles

Crude cardiac microsomal vesicles containing fragmented sarcoplasmic reticulum (SR) were prepared as previously described for rat heart. Froehlich, J. P. et al., J. Mol. Cell. Cardiol., 10, 427-438 (1978). Pooled hearts from C57 male mice sacrificed by cervical dislocation were placed in 0.9% saline on ice, trimmed of atrial and connective tissue, and weighed. The finely minced heart muscle was homogenized in 10 mM NaHCO₃ using a Polytron blender and the SR vesicles were separated from the myofilamnents, mitochondria and nuclear membranes by differential centrifugation at 8,500 and 45,000×g. SR vesicles suspended in 0.25 M sucrose +10 mM MOPS, pH 7.0 were frozen and stored in liquid nitrogen prior to use. Twenty minutes prior to measuring Ca²⁺ uptake, cardiac SR vesicles (1 mg/ml in storage buffer) were incubated with 250 μM AS delivered from a freshly-prepared 10 mM stock solution of AS (Na₂N₂O₃) dissolved in 10 mM NaOH. After dilution of the SR membranes in the Ca²⁺ uptake buffer, the change in kinetic behaviour resulting from exposure to AS was seen after a delay of ˜15 min and remained in effect for the duration of the experiment (45-60 min). Aging of the stock AS solution led to a complete loss of stimulatory activity, reflecting the decomposition of HNO to biochemically-inert products, e.g., nitrite. Stopped-flow mixing was used to measure the initial time course of Ca²⁺ accumulation by murine cardiac SR vesicles using the Ca²⁺ indicator dye, arsenazo III. Membrane vesicles (0.4 mg/ml) suspended in a medium containing 100 mM KCl, 1 mM MgCl₂, 50 μM arsenazo III, 5 mM sodium azide, and 20 mM MOPS, pH 7.4, were mixed with an equal volume of an identical medium containing 1 mM Na₂ATP at 24° C. in a manually-operated stopped-flow apparatus (Applied Photophysics, Ltd.). The change in [Ca²⁺] in the mixing cuvette was monitored using a single-beam UV-VIS spectrophotometer (AVIV, Model 14DS) with a monochromator setting of 650 nm. The total [Ca²⁺] in the uptake medium was 0.5 μM, yielding a free [Ca²⁺] in equilibrium with the Ca-arsenazo III complex of 0.2 μM (K_{A}=3.3×10⁴ M⁻¹). Spectral scans of arsenazo III conducted at different Ca²⁺ concentrations (0-30 μM) in the presence of 10 μM thapsigargin to prevent cardiac SR Ca²⁺ uptake revealed an absorbance peak for Ca²⁺ at 650 nm and an isosbestic point at 693 nm that was red-shifted from the value obtained in the absence of protein (685 nm). The addition of 250 μM AS to the incubation medium had no affect on the spectral characteristics of arsenazo III or its response to Ca²⁺. The time-dependent decrease in absorbance at 650 nm, reflecting Ca²⁺ uptake by the SR vesicles, was monitored for 30-60 s at 0.1 s intervals. Ca²⁺ dissociation from the Ca²⁺-arsenazo III complex was >100 times faster (˜60 s⁻¹) than the rates of Ca²⁺ accumulation measured in these experiments, excluding rate-limitation by the dye. The signal change due to vesicle light scattering was evaluated from separate measurements conducted under identical conditions at the isosbestic wavelength of 693 nm. For evaluation of the time course of Ca²⁺ uptake, a representative trace at 693 nm was subtracted from each of the individual traces at 650 nm acquired under identical conditions. The kinetic and thermodynamic parameters for Ca²⁺ uptake were evaluated by fitting stopped-flow signals to one- and two-exponential decay functions plus a residual term using non-linear regression (Prism, Version 3.03). Residual plots of the difference between the fitted curve and data points were used to evaluate systematic errors in the fits and to calculate the sum-of-squares error used in selecting the best fit.

### Results

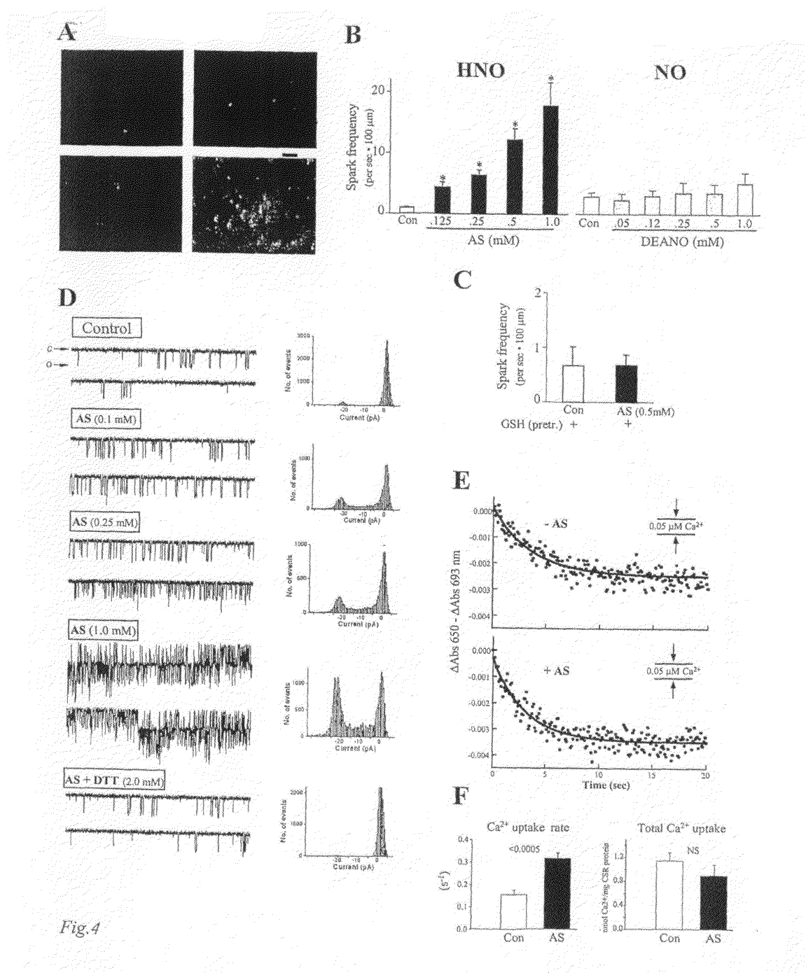

In intact myocytes, AS enhanced RyR2 opening probability, as revealed by an increased frequency of Ca²⁺ sparks assessed by line scan confocal microscopy (FIG. 4A), in a dose dependent manner (FIG. 4B left panel; 18-fold rise in spark frequency at 1 mM AS, n=10-24, p<0.001). In contrast, DEA/NO had no effect on spark generation (FIG. 4B, right panel). Individual spark amplitude, rise time, and spatial width, were unaltered by AS, indicating a primary effect on RyR2 activation. SR Ca²⁺ store depletion by thapsigargin (10 μM, 30 min) or ryanodine exposure (10 μM) abolished Ca²⁺ sparks in control and AS (0.5 mM, data not shown). The influence of AS on Ca²⁺ sparks was thiol sensitive. Preincubating cells with reduced glutathione (3 mM for 4 hr) prior to AS exposure prevented increased spark frequency (FIG. 4C), indicating that increased intracellular thiol content effectively quenched HNO signalling/action.

To further test whether HNO directly interacted with RyR2 proteins to increase open probability, purified reconstituted RyR2 were expressed in planar lipid bilayers and steady-state activity recorded with or without AS. The cis (cytosolic) solution contained 10 μM activating Ca²⁺ and recordings were made at positive 30 mV holding potential. AS (0.1 to 1 mM) produced a dose-dependent rapid increase in frequency and the mean time of open events without altering unitary channel conductance (FIG. 4D). The probability of the channel being open (Po) increased from an average 0.16±0.03 without AS to 0.46±0.07 at 0.3 mM AS added to the cytoplasmic side of the channel (n=4). This was reversible upon addition of 2 mM DTT (0.11±0.04). These findings support direct HNO-RyR2 interaction likely via a reversible reaction with thiol groups in the protein.

To test whether HNO directly enhances SR Ca²⁺ uptake, its effects on SR membrane vesicles isolated from pooled C57/B16 mouse hearts were studied. Crude SR microsomal vesicles were incubated with 250 μM AS prior to measuring ATP-dependent Ca²⁺ uptake by stopped-flow mixing at 24° C. Arsenazo III, a mid-range Ca²⁺ indicator, was used to monitor Ca²⁺ removal from the extravesicular compartment and buffer the free [Ca²⁺] at a level producing half-saturation of the Ca²⁺ pump (˜0.2 μM). Time dependent changes in absorbance at 693 nm (isosbestic wavelength) were subtracted from changes recorded at 650 nm, the absorption maximum for the Ca²⁺-arsenazo III complex. Ca²⁺ accumulation exhibited a monophasic time course with >90% of uptake occurring within the initial 20 s (FIG. 4E, upper panel). Uptake was abolished by 10 μM thapsigargin, while pre-incubation with A23187 (5 μg ionophore/mg SR protein) decreased total Ca²⁺ uptake by >50% reflecting partial collapse of the transport gradient (data not shown).

AS/HNO exposure increased the rate constant for Ca²⁺ uptake by 104% based on exponential analysis of the 650-693 nm signal (0.1563 s⁻¹ vs. 0.3204 s⁻¹; p<0.0005; n=6) (FIG. 4E lower panel and FIG. 4F). There was no difference in total Ca²⁺ uptake at equilibrium (from 0.00257±0.0003 to 0.00202±0.000 μM, before and after AS exposure, respectively; n=6; p=NS), implying that activation by HNO increases the catalytic efficiency of the Ca²⁺ pump without changing its thermodynamic efficiency. No stimulation of SR Ca²⁺ uptake activity was obtained following exposure to a test solution of AS that had decayed completely to products, e.g., nitrite (data not shown). The enhanced SERCA2a function, and unaltered net SR Ca²⁺ uptake in these vesicle experiments are consistent with the AS-induced enhancement of SR-dependent [Ca²⁺]ᵢ decay and SR Ca²⁺ leak in intact myocytes (FIGS. 3H-K and 4A-D).

In the physiologic setting, cardiac contractile force and rate of force decay are typically enhanced via cAMP/PKA coupled mechanisms that trigger activator Ca²⁺ to stimulate the myofilaments. HNO is very different, as it augments cardiac contractility and relaxation independent of cAMP/PKA, modulating the Ca²⁺ transient by direct enhancement of SR Ca²⁺ uptake and release. These two counterbalancing effects likely explain why there is no net rise in diastolic Ca²⁺ or change in total SR Ca²⁺ load. Increased SR Ca²⁺ release with unaltered total SR Ca²⁺ content suggests AS has an effect on RyR2 function, rather than inducing a leak secondary to increased intra-SR Ca²⁺ stores. Kubalova, Z. et al., Proc. Natl. Acad. Sci. USA, 102, 14104-14109 (2005). Moreover, this direct effect is redox sensitive and reversible.

The action of HNO on RyR2 is quite different from that exerted by NO donors, β-agonists and caffeine. NO donors have been reported to enhance (Stoyanovsky, D. et al., Science, 279, 234-237 (1998)) or inhibit RyR2 (Zahradnikova, A. et al., Cell Calcium, 22, 447-454 (1997)), and reportedly do not increase basal Ca²⁺ spark frequency (Ziolo, M. T. et al., Am. J. Physiol Heart Circ. Physiol., 281, H2295-H2303 (2001)). β-adrenergic agonists stimulate RyR2 open probability via PKA-mediated phosphorylation. Hain, J. et al., J. Biol. Chem,. 270, 2074-2081 (1995). Thus, without being limited to any theory, it is believed that resting Ca²⁺ spark frequency can increase during β-adrenergic stimulation by PKA-mediated phosphorylation of both RyR2 (to increase Pₒ probability) and PLB (to increase SR Ca²⁺ load). Zhou, Y. Y. et al., J. PhysioL, 52, 351-361 (1999). In transgenic mice overexpressing human β₂Ars, Ca²⁺ sparks are larger and more frequent than in non-transgenic cells, despite having resting cytosolic Ca²⁺ and Ca²⁺ SR load similar to controls. Id. This suggests that β-mediated cAMP-PKA activation not only alters RyR2 sensitivity to Ca²⁺ but also the Ca²⁺ release-linked RyR2-inactivation (Sham, J. S. et al., Proc. Natl. Acad. Sci. USA, 95, 15096-15101 (1998)), potentially changing SR stability. In stark contrast, HNO increased spark frequency without altering individual spark characteristics, and did not adversely impact Ca²⁺ stability. HNO action on RyR2 is also distinct from that of caffeine. It has been reported that in isolated mouse myocytes, caffeine increases the frequency of spontaneous Ca²⁺-release events (Ca²⁺ waves) that is maintained even after discontinuation of the drug (Balasubramaniam, R. et al., Am. J Physiol., 289, H1584-H1593 (2005)) and significantly reduces SR Ca²⁺ content.

The unique action of HNO on RyR2 may be explained by HNO thiophilic chemistry. HNO effects on RyR2 were promptly reversed by reducing equivalents, suggesting real-time competition for HNO between free thiols and critical structural thiol residues on the RyR2. This is in keeping with the data at the whole myocyte level in which a 6% increase in intracellular GSH blunted 57% of the HNO effect on sarcomere shortening, suggesting HNO “selective” targeting of thiolate (—S⁻) residues of RyR2 rather than a more generalized thiol involvement. Identification of these specific targets awaits sub-proteome analysis of cysteine modification, with site mutagenesis to identify the functional importance of particular targets.

In order to enhance and sustain cardiac inotropy, it has been suggested that the velocity of Ca²⁺ re-uptake into the SR during relaxation should ideally increase (Diaz, M. E. et al., Cell Calcium, 38, 391-396 (2005)), and HNO also achieved this effect. While the rate increased, total Ca²⁺ uptake did not change, implying that thermodynamic efficiency of the Ca²⁺ pump was unchanged by HNO. This implies that HNO works by increasing the catalytic efficiency of the pump, although the mechanism by which this occurs is presently unknown. It is also possible that the enhanced uptake activity of SERCA2a counterbalances greater Ca²⁺ release and that blocking the latter (e.g., with ruthenium red) would increase net Ca²⁺ uptake. The enhanced Ca²⁺ uptake activity with AS/HNO is reminiscent of the stimulation observed in ER microsomes from Sf21 cells expressing SERCA2a in the absence of phospholamban (Mahaney, J. E. et al., Biochemistry, 44, 7713-7724 (2005)), and AS/HNO may also target PLB to relieve its inhibition of SERCA2a. Efforts are underway to clarify these mechanisms.

The present findings lend strong support to prior intact animal data (Paolocci, N. et al., Proc. Natl. Acad. Sci. USA, 98, 10463-10468 (2001); Paolocci, N. et al., Proc. Natl. Acad. Sci. USA, 100, 5537-5542 (2003)) showing the ability of AS to improve cardiac function in intact failing hearts, independent of β-adrenergic blockade, and additive to beta-adrenergic agonists. Its mechanism, a reversible, thiol-dependent, direct enhancement of SR Ca²⁺ uptake and release, is novel and may be unique to HNO. Evidence of the thiophilic nature of HNO suggests it may indeed be an in vivo signalling molecule (Schmidt, H. H., et al., Proc. Natl. Acad. Sci. USA, 93, 14492-14497 (1996); Adak, S. et al., J. Biol. Chem., 275, 33554-33561 (2000)), although methods to test this hypothesis are currently unavailable. Exploration of HNO biological activity is in its infancy, but the current findings suggest novel modulating effects on the heart with potential utility for cardiac failure treatment as well as potential impact on other cellular systems that heavily rely on intracellular Ca²⁺ cycling for their basal and agonist-stimulated function.

### EXAMPLE 5

### Effect of HNO/NO⁻ on Cardiac Function in Normal and Failing Canine Myocardium

The effect of AS on Ca-ATPase partial reactions and Vmax was measured in sealed cardiac sarcoplasmic reticulum (CSR) membrane vesicles isolated from normal (N) and failing (F) (tachy-pacing-induced) dog hearts. Spontaneous E2P hydrolysis measured by chasing phosphorylated SERCA2a with 5 mM EGTA obeyed slow, monophasic kinetics in N and F CSR vesicles (12 s⁻¹ vs. 11 s⁻¹), but increased significantly (76 s⁻¹ vs. 111 s⁻¹) following exposure to 0.25 mM AS. In the presence of 2.5 mM oxalate (Ca²⁺-loading conditions), 0.25 mM AS stimulated maximal Ca-ATPase activity in N and F CSR (4% vs. 9% compared to control). Vmax stimulation increased without oxalate (27% in N CSR) and was abolished by the Ca²⁺ ionosphere, A23187. The results suggest that HNO/NO activates SERCA2a in N and F CSR by activating E2P hydrolysis, which competes with Ca²⁺ binding to the luminal transport sites on E2P. This relieves back inhibition of SERCA2a by the Ca²⁺ transport gradient, increasing Vmax. These HNO/NO effects resemble changes in SERCA2a activity following the relief of phospholamban (PLB) inhibition, suggesting that they result from covalent modification of PLB, SERCA2a, or both.

The results show that HNO/NO⁻ generated by AS has positive inotropic and lusitropic effects on cardiac function in normal and failing canine myocardium, implicating activation of the cardiac sarcoplasmic reticulum (CSR) Ca²⁺ pump (SERCA2a).

### EXAMPLE 6

### Effect of Thiol and Guanylate Cyclase Inhibition on AS Inotropy

Nitroxyl (HNO) confers positive inotropy in vivo. Here, it was determined whether HNO action stems from a direct influence on sarcoplasmic reticulum (SR) Ca²⁺ cycling, involving enhanced Ca²⁺ release from ryanodine receptors (RyR2). Myocytes were isolated from ST mice, suspended in Tyrode's solution (1 mM Ca²⁺) and field stimulated (0.5 Hz, 25° C.). Sarcomere shortening (SS) was assessed by real-time image analysis, Ca²⁺ transients from Indo-1 fluorescence. RyR2 activity was determined by optical imaging of Ca²⁺ release from single Ca²⁺ release units. The HNO donor Angeli's Salt (AS) induced dose-dependent inotropy (SS: 73±31% at 0.5 mM; 131±31% at 1 mM; all n=15, p<0.05 vs. base; <0.1 mM: no effect). In contrast, the NO donor DEA/NO reduced SS by 55-65% at 5-50 μM (both p<0.05 vs. base), with no effect at higher doses. Inhibition of guanylate cyclase (ODQ, 10 μ, 30′) fully blocked DEA/NO negative inotropy but had no effect on AS action (157±40%; n=15, p=NS vs. AS 1 mM). However, co-infusion with the thiol-donating compound N-acetyl-L-cysteine (NAC, 3 mM) abolished AS inotropy. A rapid infusion of caffeine demonstrated that SR Ca²⁺ stores declined with 1 mM AS (%[Ca²⁺]ᵢ: 138±17 vs. 223±34, n=8; p=0.05 vs. caffeine alone). Accordingly, AS/nitroxyl increased frequency of calcium sparks (CSF, unitary SR release): at 0.5 mM AS, CSF was almost 7 times higher than in controls (26±3 vs. 4±1 sparks/100 μm/s, respectively, p<0.01. Myocyte pre-treatment with DSH (w.5 mM for 3 hrs) abrogated AS-induced increase in CSF. Equimolar doses of DEA/NO did not significantly affect CSF. Furthermore, co-treatment with the SR Ca²⁺ uptake blocker thapsigargin (3 μM) blunted AS inotropy (52±14%, p<0.05 vs. AS, n=16). HNO in vitro inotropy is cGMP-independent and due to the activation of RyR2 to release calcium. Increasing intracellular thiol concentration prevents HNO effects, likely through competition with thiol residues located on RyR2.

The results show that nitroxyl increases calcium release from ryanodine receptors in a thiol-sensitive but cGMP-independent manner.

### EXAMPLE 7

### HNO/NO⁻ Action on SERCA2a Function and Sensitivity to Intracellular Thiol Content in Isolated Murine Cardiomyocytes

Nitroxyl (HNO) donors are redox-sensitive positive inotropes in vivo, although mechanism of action has remained unclear. Here, the results show that HNO directly stimulates sarcoplasmic reticular (SR) Ca²⁺ release and uptake, in a manner that is sensitive to the intracellular levels of reducing equivalents. In isolated murine cardiomyocytes, the HNO donor Angeli's Salt (AS) increase sarcomere shortening (SS, e.g. 117±25% at 1.0 mM, n=21;p<0.01 vs. base) without changes in Ca²⁺ transients, an effect that was not reproduced by equimotar NO donated by DEA/NO. Inhibition of guanylyl-cyclase or PKG did not alter HNO response. To check for HNO sensitivity to intracellular thiol content, myocyte thiol quantitation was performed by two-photon microscopy. Pre-incubation with reduced glutathione (GSH, 4 mM for 3 hrs) increased intracellular thiol content (+6%, p<0.05, n=40) and HNO response was cut by half: SS: 58±19%, n=14, p=0.05 vs. 1 mM AS alone). To assess for HNO action on cardiac ryanodine receptors (RyR2), Ca²⁺ sparks were analyzed by optical imaging, and RyR2 were reconstituted in planar lipid bilayers to perform single channel recording. HNO increased frequency of calcium sparks (CSF) in a dose-dependent manner: with a 7-fold increase at 0.5 mM AS (26±3 vs. 4±1 sparks/100 μm/s, p<0.01). Pre-treatment with GSH abrogated the increase in CSF. In reconstituted RyR2, HNO produced an acute increment in the frequency/mean time of open vents without altering the unitary conductance. The open probability of the channel (Po) increased from 0.16±0.03 (control) to 0.25±0.05, 0.46±0.07 and 0.69±0.11 after adding 0.1, 0.3, and 1.0 mM AS to the cytosplasmic (cis) side of the channel. Po of AS-activated channels reverted to control after adding 2 mM of the sulfhydril reducing agent DTT to the cis side (0.11±0.04). Finally, to test whether HNO affects SERCA2a function, AS (250 μM) was added to isolated cardiac mouse SR vesicles. HNO enhanced the rate of initial Ca²⁺ uptake. Thus, HNO increases myocyte contractility (positive inotropy) and speeds relaxation (positive lusitropy) through potent activation of RyR2 and to Ca²⁺ SR uptake kinetics, respectively. These properties may contribute to the beneficial action of HNO-releasing compounds in heart failure.

The results show that HNO/NO⁻ enhances SR Ca²⁺ release and uptake in murine cardiomyocytes.

### EXAMPLE 8

### Effect of HNO/NO⁻ on Contractility in Murine Myocytes

Nitroxyl anion (HNO/NO⁻) donors have been shown to exert similar positive inotropic/lusitropic effects in normal and failing hearts in vivo that are not reproduced by NO/nitrate donors. In vivo HNO infusion appears to be coupled to calcitonin gene-related peptide (CGRP) systemic release. However, differently from HNO, CGRP positive inotropy may be sensitive to β-blockade and severely blunted in CHF hearts. It is hypothesized that the HNO/NO⁻ donor Angeli's Salt (AS) has a direct positive inotropic effect on myocyte contractility in Gαq overexpressing mice, a well established model of hypertrophy and cardiac failure.

Cardiac myocytes were isolated from WT and Gαq overexpressing 2-6 month old FVB/N mice, suspended in Tyrode's solution (1 mM calcium) and field stimulated at 0.5 Hz at 23° C. Sarcomere shortening (SS) was assessed by real-time image analysis; data are presented at steady-state (10 minutes drug infusion).

Cardiomyocytes from Gαq overexpressing mice exhibited a depressed response to isoproterenol (ISO). In particular, at 2.5 and 10 nM, ISO did not elicit any contractile response, while in WT cells the same ISO concentrations enhanced SS by 74±24% and 250±75%, respectively (both p<0.05 versus baseline and versus Gαq, n=6). In stark contrast, Gαq myocytes were still sensitive to direct stimulation of adenylyl cyclase through the infusion of forskolin (FSK), in a dose dependent manner. SS increased by 85±9% with 25 nM FSK and 158±62% with 100 nM FSK (both p<0.05 versus baseline, n=6), with no differences compared to control cells, a profound β-adrenergic desensitization. Interestingly, in Gαq myocytes, AS infusion showed a positive inotropic effect which was not significantly different from WT cells. At 250 μM, AS produced an increase in SS of 22±11% while at 500 μM such increase was 40±11% (both p<0.05 versus baseline, n=10).

Cardiomyocytes from Gαq overexpressing mice exhibit a profound β-adrenergic desensitization. On the other hand, nitroxyl still exerts a positive inotropic effect, which appears to be independent from the β-adrenergic signaling pathway. Hence, nitroxyl action might be clinically relevant as a therapeutical strategy in the treatment of heart failure. Thus, the results show that HNO/NO⁻ increases contractility at mycocytes level in a murine model of cardiac contractile failure.

### EXAMPLE 9

### End-Systolic and End-Diastolic Pressure-Dimension Assessment

Adult male mongrel dogs (22-25 kg) were chronically instrumented for pressure-dimension analysis as described. See, Paolocci et al., “Positive Inotropic and Lusitropic Effects of HNO/NO⁻ in Failing Hearts: Independence from Beta-Adrenergic Signaling,” Proc. Natl. Acad. Sci. USA., 100, 5537-5542 (2003); and Senzaki et al., Circulation, 101, 1040-1048 (2000). Animals were anesthetized with 1% to 2% halothane after induction with sodium thiopental (10-20 mg/kg, i.v.). The surgical/experimental animal protocol was approved by the Johns Hopkins University Animal Care and Use Committee. The surgical preparation involved placement of a LV micromanometer (P22; Konigsberg. Instruments, Pasadena, Calif.), sonomicrometers to measure anteroposterior LV dimension, an inferior vena caval perivascular occluder to alter cardiac preload, aortic pressure catheter, ultrasound coronary-flow probe (proximal circumflex artery), and epicardial-pacing electrodes for atrial pacing. Cardiac failure was induced by rapid ventricular pacing for 3 weeks as described. See, Paolocci et al., supra, and Senzaki et al., supra.

Hemodynamic data were digitized at 250 Hz. Steady-state parameters were measured from data averaged from 10-20 consecutive beats, whereas data collected during transient inferior vena cava occlusion were used to determine pressure-dimension relations. These relations strongly correlate with results from pressure-volume data in normal and failing hearts, as previously validated. Cardiovascular function was assessed by stroke dimension, fractional shortening (stroke dimension/end-diastolic dimension [EDD]), estimated cardiac output (stroke dimension×HR), peak rate of pressure rise (dP/dtₘₐₓ), end-systolic elastance (Eₑₛ, slope of end-systolic pressure-dimension relation [ESPDR]), the slope of dP/dtₘₐₓ-EDD relation (D_{EDD}) (see, Little, Circ Res., 56:808-815 (1985)), pre-recruitable stroke work (PRSW), (based on dimension-data), estimated arterial elastance (Ea, end systolic pressure/stroke dimension) and estimated total resistance (RT, stroke dimension×HR/mean Aortic pressure). Eₑₛ, D_{EDD} and PRSW provide load-insensitive contractility measures.

The end-diastolic pressure-volume relationship (EDPVR) was determined applying non-linear regression analysis to the end-diastolic pressure and volume points (P_{ed} and V_{ed}, respectively), according to Kass, Cardiol Clin., 18, 571-86 (2000)(Review). These data were fit to the following two equations P_{ed}=Pₒ+be^{aVed} and P_{ed}=be^{aVed} (the second expression simply eliminating the Pₒ term). The former equation is preferred as it does not presume a zero-pressure decay asymptote.

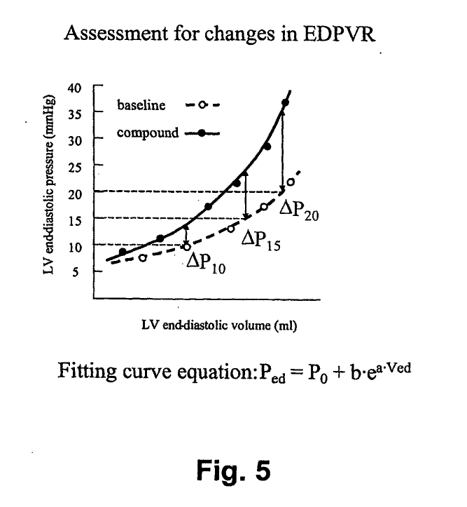

In order to evaluate the impact of each pharmacological intervention on the EDPVR, changes in end-diastolic pressure from baseline (ΔP_{ed}) at volumes providing baseline end-diastolic pressure of 10, 12.5, 15, 17.5 and 20 mmHg EDP (V₁₀, V₁₅, V₂₀, respectively) were determined (FIG. 5).

### Effects of HNO, NO and Nitrate Donors on EDPVR

It is estimated that 30% to 50% of heart failure patients have preserved systolic left ventricular (LV) function, often referred to as diastolic heart failure (DHF). This appears to occur more prominently in patients that are elderly, hypertensive, female, and have hypertension. Mortality is high in these patients, and morbidity and rate of hospitalization are similar to those of patients with systolic heart failure. (See, Kass et al., “What Mechanisms Underlie Diastolic Dysfunction in Heart Failure?” Circ. Res., 94(12):1533-42 (Jun. 25, 2004).) The management of patients with diastolic heart failure is essentially empirical, limited, and disappointing. New drugs, devices, and gene therapy based treatment options are currently under investigation. See, Feld et al., 8(1), 13-20 (2006).

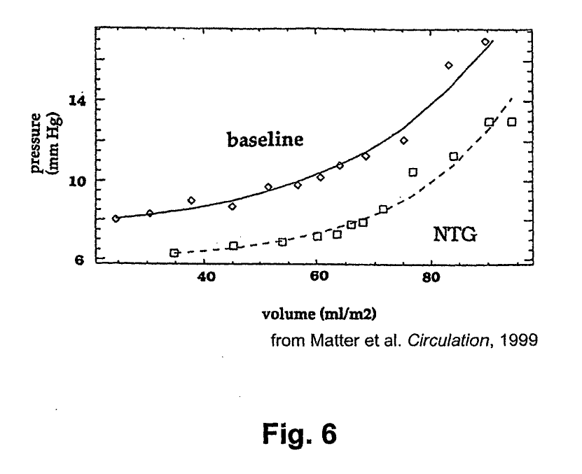

It has been reported that nitric oxide donors may improve diastolic function (see, Paulus et al., Heart Fail. Rev., 7(4), 371-83 (October 2002)). However, as shown in FIG. 6 with nitroglycerin, such amelioration consists of a parallel downward shift of the EDPVR relation (see, Matter et al., Circulation, 99(18), 2396-401 (1999)), likely reflecting an unloading effect exerted by the NO/nitrate donor on the heart. In contrast, changes in the slope of the EDPV relation, different from parallel shift, would be expected (particularly at the highest end-diastolic volumes/pressures) if left-ventricle compliance (distensibility) is really affected.

Previous studies suggest that HNO donors may improve myocardial relaxation in CHF conscious preparation as well as lower diastolic pressure (see, Paolocci et al., supra). Yet, EDPVR analysis has never been perforned.

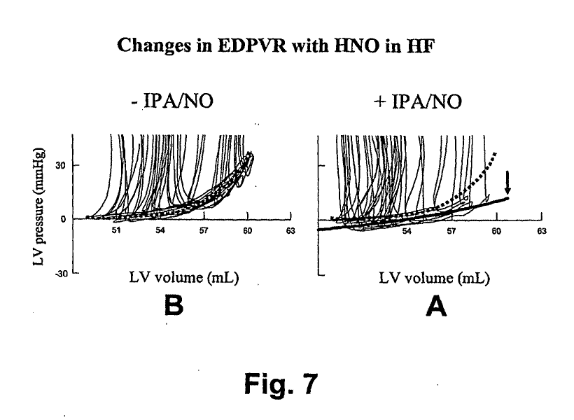

As shown in FIG. 7, the results demonstrate that HNO donated by IPA/NO is able to produce a downward shift of the EDPVR in CHF preparations, indicating not only an unloading effect on the heart, but more importantly a change in the slope of the EDPVR. The arrow shows that at the higher filling volumes diastolic pressure is less in hearts treated with IPA/NO versus untreated CHF hearts.

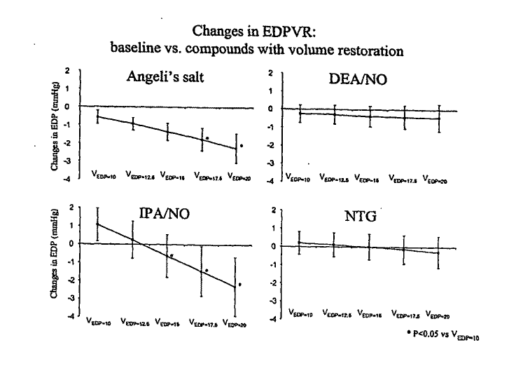

FIG. 8 shows mean changes in ΔP_{ed} at the specified volumes. All in all, these changes were relatively small. Yet, in the case of HNO donors, both IPA/NO and AS (data not shown), the EDPVR declined significantly from baseline curve-fitting, likely indicating an improvement in left-ventricular compliance. In contrast, neither NO (from DEA/NO) nor nitrate (from NTG) significantly improved LV compliance but rather induced a parallel down-ward shift of the EDPVR as illustrated for NTG in FIG. 6 due to changes in the ventricular loads.

All publications, patents and/or patent applications identified above are herein incorporated by reference.

The invention being thus described, it will be apparent to those skilled in the art that the same may be varied in many ways without departing from the spirit and scope of the invention. Such variations are included within the scope of the invention to be claimed.

We claim:

1. A method for treating diastolic dysfunction or a disease, disorder or condition associated with diastolic dysfunction, comprising:
(i) identifying a subject in need of treatment for diastolic dysfunction or a disease, disorder or condition associated with diastolic dysfunction; and
(ii) administering an effective amount of a nitroxyl donor to the subject.

2. The method of claim 1, wherein the nitroxyl donor is an S-nitrosothiol compound.

3. The method of claim 1, wherein the nitroxyl donor is a thionitrate compound.

4. The method of claim 1, wherein the nitroxyl donor is a hydroxamic acid or a pharmaceutically acceptable salt thereof.

5. The method of claim 1, wherein the nitroxyl donor is a sulfohydroxamic acid or a pharmaceutically acceptable salt thereof.

6. The method of claim 1, wherein the nitroxyl donor is Piloty's acid.

7. The method of claim 1, wherein the nitroxyl donor is isopropylamine diazeniumdiolate (IPA/NO).

8. The method of claim 1, wherein the nitroxyl donor is Angeli's salt.

9. The method of claim 1, wherein the subject is receiving beta-adrenergic receptor antagonist therapy.

10. The method of claim 1, wherein the disease, disorder or condition is diastolic heart failure.

11. The method of claim 1, wherein the subject is hypertensive.

12. The method of claim 1, wherein the subject is diabetic.

13. The method of claim 1, wherein the subject has metabolic syndrome.

14. The method of claim 1, wherein the subject has ischemic heart disease.

15. The method of claim 1, wherein the subject is elderly.

16. The method of claim 1, wherein the subject is female.

17. A method for treating heart failure, comprising:
(i) identifying a subject who is experiencing and/or is predisposed to impaired SR Ca²⁺ release and/or uptake, and in need of treatment for heart failure; and
(ii) administering an effective amount of a nitroxyl donor to the subject.

18. A method for modulating SR Ca²⁺ release and/or uptake, comprising administering an effective amount of a nitroxyl donor to a subject in need of modulation of SR Ca²⁺ release and/or uptake.

19. A method for enhancing myocyte relaxation, preload or E2P hydrolysis, comprising administering an effective amount of a nitroxyl donor to a subject in need of enhancement of myocyte relaxation, preload or E2P hydrolysis.

20. The method of claim 19, wherein the preload is measured by end-diastolic volume (EDV) or end-diastolic pressure (EDP).

21. A method for treating ventricular hypertrophy, comprising administering an effective amount of a nitroxyl donor to a subject in need of treatment of ventricular hypertrophy.
